(19) United States
(12) Patent Application Publication
(10) Pub. No.: US 2026/0232494 A1
(43) Pub. Date: Aug. 13, 2026

(54) A NEGATIVE PRESSURE WOUND THERAPY DRESSING

(71) Applicant: Mölnlycke Health Care AB, Mölndal (SE)

(72) Inventors: Anette Svensson Henriksson, Mölndal (SE); Peter Madlung, Göteborg (SE)

(21) Appl. No.: 19/137,358

(22) PCT Filed: Dec. 28, 2022
(86) PCT No.: PCT/EP2022/087937

§ 371 Date: Jun. 10, 2025

(51) Int. Cl. A61F 13/05; A61F 13/0246; A61F 13/06; A61F 13/512

(52) U.S. Cl. CPC A61F 13/05; A61F 13/0253; A61F 13/064; A61F 13/512

(57) ABSTRACT

Described is a NPWT dressing that includes a backing layer, an adhesive skin contacting layer and an absorbent pad arranged between the backing layer and the adhesive skin contacting layer. The backing layer comprises a coupling site configured to connect the dressing to a negative pressure source. The absorbent pad has a pad outer edge(s). The backing layer and the adhesive skin contacting layer extends beyond the pad outer edge(s) forming a border portion around the absorbent pad. The adhesive skin contacting layer has perforations, the perforations forming a first perforated area extending over the absorbent pad allowing passage of liquid from a wound into the absorbent pad. The absorbent pad has a non-rectangular shape, and the first perforated area forms a rectangular shape.

### TECHNICAL FIELD

The present disclosure generally relates to a negative pressure wound therapy dressing. More specifically, the present disclosure relates to a negative pressure wound therapy dressing comprising an adhesive skin contacting layer comprising perforations.

### BACKGROUND

Negative pressure wound therapy (NPWT) is a method of drawing out fluid and infection from a wound to promote healing of the wound by applying a sub-atmospheric pressure to the wound using a negative pressure pump. By drawing out excess wound exudate from the wound site, blood flow to the area increases, which promotes the formation of granulation tissue.

Wound dressings intended for use in negative-pressure therapy generally comprises a liquid-impermeable backing layer, a wound-facing film layer provided with openings and an absorbent layer arranged between the backing layer and a wound-facing film layer. During operation, wound exudate can be removed from the wound, with the liquid being transported through the openings of the wound-facing film.

NPWT dressings may be intended for specific types of wounds, such as for example diabetic related ulcers, and there may be a desire to have specifically shaped wound pads designed to fit with the intended type of wound.

NPWT dressings furthermore comprise an adhesive edge for air-tight closure of the wound area, which is of particular importance for such dressings. For some of the wound dressings the wound facing film is a skin adhesive skin contacting layer. The skin contacting layer may be co-extensive with the backing layer and provided with openings.

### SUMMARY

It is an object of the present disclosure to make available an improved negative pressure wound therapy (NPWT) dressing, which is adapted to specific types of wounds, is convenient and cost efficient to manufacture and has maintained or improved wound healing capabilities. This is achieved by a negative pressure wound therapy dressing according to claim 1.

A NPWT dressing as disclosed herein comprises a backing layer, an adhesive skin contacting layer and an absorbent pad arranged between the backing layer and the adhesive skin contacting layer. The backing layer comprises a coupling site configured to connect the dressing to a negative pressure source. The absorbent pad has a pad outer edge(s). The backing layer and the adhesive skin contacting layer extends beyond the pad outer edge(s) forming a border portion around the absorbent pad. The adhesive skin contacting layer comprises perforations, the perforations forming a first perforated area extending over the absorbent pad allowing passage of liquid from a wound into the absorbent pad. The absorbent pad has a non-rectangular shape, and the first perforated area forms a rectangular shape having a first, a second, a third and a fourth corner portion.

The NPWT dressing is a dressing comprising an absorbent pad having a non-rectangular shape, such as a circular, oval or any other non-rectangular form. The absorbent pad may comprise two or more interlinked shaped sections forming a non-rectangular shape.

According to the present disclosure, the adhesive skin contacting layer is provided with perforations and an absorbent pad arranged between the backing layer and a wound-facing film layer. During operation, wound exudate and infection can be removed from the wound, with the liquid being transported through the perforations of the perforated adhesive skin contacting layer thereby promoting healing of the wound.

Typically, perforations are made across the whole skin contacting surface of NPWT dressings. Furthermore, perforation tools generally provide perforation patterns having square or rectangular shapes which matches well with absorbent pads having square and rectangular shapes. However, for absorbent pads having non-rectangular shapes, as disclosed herein, the nonmatching shape of the perforation tools may have a negative impact on the liquid draining capacity of the NPWT and/or on the sealing between the skin of the patient and the border region surrounding the absorbent pad. The adhesive skin contacting layer may be perforated to form the perforated area by means an ultrasonic device e.g. as disclosed in the International patent publication No. WO2007/069990A1, Method for perforating heat meltable material, in the name of Mölnlycke Health Care AB.

The present disclosure is at least partially based on the identification of the problem that the arrangement of a perforated area having a rectangular shape needs to be adjusted in respect to the absorbent pad for maintaining a high liquid draining capacity while preserving the integrity of the border portion and not compromising an air-tight sealing of the border portion to the skin.

The negative pressure source may comprise a housing, a negative pressure pump arranged within the housing and a canister detachably connected to the housing. A tubing assembly may be configured to fluidly connect the NPWT dressing to the NPWT device.

The tubing assembly may comprise a first tubing configured to be connected to the dressing, wherein a distal end of the first tubing is attached to a first connector portion arranged at the coupling side and a second tubing configured to be connected to the canister, wherein a distal end of the second tubing is attached to a second connector portion configured to be detachably connected to the first connector portion, wherein the system comprises means to supply air to the dressing.

The absorbent pad outer edge(s) may have a first outer edge portion and a second outer edge portion, the first and the second outer edge portion being opposing outer edge portions and wherein the first perforated area extends from the first outer edge portion to the second outer edge portion of the absorbent pad, or at least extends from a respective first and second point, at a distance of 10 mm or less from the first and the second outer edge portion respectively, preferably 7 mm or less from the first and the second outer edge portion respectively.

With “outer edge portion” herein is meant a portion of the outer edge of the absorbent pad.

Optionally, the first corner portion of the first perforated area is arranged at the first outer edge portion of the absorbent pad and the fourth corner portion is arranged at the second outer edge portion of the absorbent pad, or at least the respective first and the fourth corner portion is arranged at a distance of 10 mm or less from the first and the second outer edge portion respectively, preferably at a distance of 7 mm or less from the first and second outer edge portion respectively.

Such positioning of the first perforated area with respect to the absorbent pad improves the liquid draining capacity of the absorbent pad and thus promotes an enhanced wound healing.

The pad outer edge(s) may have a first outer edge portion and a second outer edge portion, the first and the second outer edge portion being opposing outer edge portions. The first perforated area may extend from the first outer edge portion to the second outer edge portion of the absorbent pad and wherein the first perforated area extends into the border portion around the absorbent pad with a greatest distance di between an outmost point of the first perforated area and the pad outer edge(s) being within the range of from 0.5 mm and 10 mm.

The first perforated area may have a first, a second, a third and a fourth corner portion and the first corner portion may be arranged at the first outer edge portion of the absorbent pad and the fourth corner portion may be arranged at the second outer edge portion of the absorbent pad and wherein a greatest distance d2 from the first perforated area and the pad outer edge(s) is within the range of from 0.5 mm to 15 mm, as measured from a point arranged between the first and the second corner portions, preferably within the range of from 0.5 mm to 10 mm, as measured from a point arranged between the first and the second corner portions. The point being taken along a border of the first perforated area between the first and the second corner portion.

A ratio of the distance d1 and the distance d2 may be within the range of from 0.2:1 to 1:1.

It has been found by the present inventors that such greatest distance d2 and such ratio between the distance d1 and the distance d2 may maintain a surprisingly high liquid draining capacity while preserving the integrity of the border portion and not compromising an air-tight sealing of the border portion to the skin.

The border portion may comprise a circumferential perforation free area extending along an outer edge of the entire NPWT dressing and wherein the circumferential perforation free area has a width (w) of at least 10 mm.

The perforations may, in the first perforated area, be arranged with a distance within the range of from 1 mm to 4 mm to an adjacent perforation, preferably with a distance within the range of from 1 mm to 3 mm to an adjacent perforation.

The perforations may each have a diameter within the range of from 0.4 mm to 2 mm.

The perforations may be evenly distributed over the entire first perforated area.

The absorbent pad may have a wound facing portion, a negative pressure source connecting portion and a bridging portion connecting the wound facing portion with the negative pressure source connecting portion, wherein the first perforated area is arranged over the first wound facing portion.

A NPWT dressing comprising such absorbent pad, may be particularly adapted for application to a foot ulcer, such a diabetic foot ulcer, with the wound facing portion being applied to an ulcer on the foot and negative pressure source connecting portion may be arranged on the leg of the patient, such as on the ankle or any lower portion of the leg.

The absorbent pad of such foot ulcer NPWT dressing has a rather particular shape and typical perforation tools are not adapted to provide perforation patterns having such matching shapes. NPWT dressings has been proven highly efficient in treating diabetic foot ulcers and the liquid draining capacity of the NPWT dressing is of utmost importance. Furthermore, such NPWT dressings being applied to a foot, ankle and lower part of the leg will be exerted to torsional forces upon movement of the foot. It is therefore important to have a high adhesion along the entire boarder portion to avoid that air passages are formed, and that air enters into the absorbent pad and affects the pressure and the liquid drainage negatively.

A wound dressing comprising such absorbent pad having a wound facing portion, a negative pressure source connecting portion and a bridging portion may have corresponding portions, i.e. meaning that the wound dressing follows the contours of the absorbent pad.

The wound facing portion may have a circular or oval shape. The wound facing portion boundary towards the bridging portion may be seen as a continuation of the circular or oval shape such as forming a complete symmetrical circle or oval.

The first perforated area may cover at least 80% of the wound facing portion.

Optionally, the bridging portion is free from perforations. This may provide enhanced adhesion along the bridging portion which may be particularly exerted to torsional forces upon movement of the foot.

Optionally, the negative pressure source connecting portion is free from perforations.

The adhesive skin contacting layer may comprise a second perforated area and wherein the second perforated area extends over the bridging portion of the absorbent pad. It is believed that this will improve the breathability of the skin at those portions as moisture evaporating from the skin can be transported away from the skin and not be occluded by the dressing to the same extent as a non-perforated areas would be.

The first perforated area may have a width w1 and a length l1 and the second perforated area may have a width w2 and a length l2. The width w1 of the first perforated area may be greater than the width w2 of second perforated area, optionally at least 2 times greater.

The absorbent pad may have an elongated non-rectangular shape and wherein the adhesive skin contacting layer may comprise a second perforated area extending over the absorbent pad allowing passage of liquid from a wound into the absorbent pad, the second perforated area having a rectangular shape. For certain elongated non-rectangular shapes of the absorbent pads it has been found advantageous to provide the adhesive skin contacting layer with a second perforated area to improve the coverage of the area of the adhesive skin contacting layer coinciding with the absorbent pad while maintaining the adhesive properties along the border portion.

The second perforated area may be arranged offset with respect to the first perforated area as seen along any transverse axis extending in a transverse direction. Such arrangement of the perforation areas may improve the coverage of the area of the adhesive skin contacting layer coinciding with the absorbent pad while maintaining the adhesive properties along the border portion.

The adhesive skin contact layer may comprise a silicone-based adhesive.

The fact that the adhesive skin contact layer is a silicone-based adhesive makes is more important to manage the ration of perforated area vs. non-perforated areas at the border of the dressing as silicone-based adhesive is very gentle and lenient to the skin and not as strong as other adhesives.

The adhesive skin contact layer may furthermore comprise a polymeric film provided with a silicone based adhesive coating.

### BRIEF DESCRIPTION OF THE DRAWINGS

The various aspects of the present disclosure, including its particular features and advantages, will be readily understood from the following detailed description and the accompanying drawings, in which:

FIG. 1 illustrates a NPWT dressing according to the present disclosure and a negative pressure source assembly, and

FIG. 2-5 illustrate exemplary NPWT dressings according to the present disclosure.

### DETAILED DESCRIPTION

The present disclosure will now be described more fully hereinafter with reference to the accompanying drawings, in which currently preferred embodiments of the present disclosure are shown. The present disclosure may, however, be embodied in many different forms and should not be construed as limited to the embodiments set forth herein; rather, these embodiments are provided for thoroughness and completeness, and fully convey the scope of the present disclosure to the skilled person. Like reference characters refer to like elements throughout.

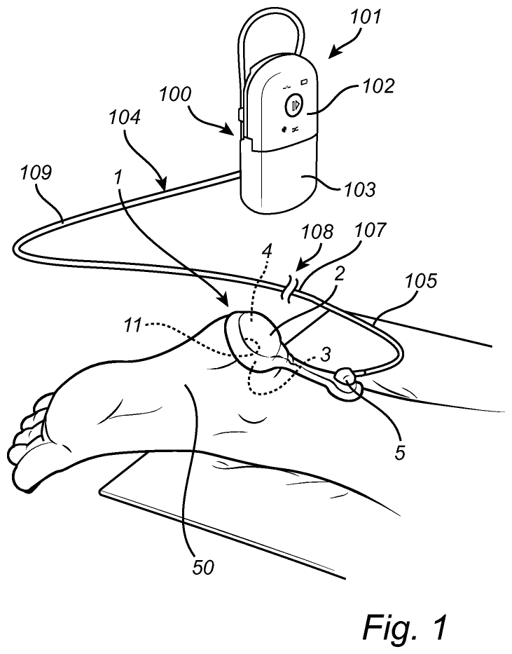

With reference to FIG. 1, a NPWT dressing 1 as disclosed herein is applied to a foot 50 of a patient. The NPWT dressing 1 comprises a backing layer 2, an adhesive skin contacting layer 3 and an absorbent pad 4 arranged between the backing layer 2 and the adhesive skin contacting layer 3. The backing layer 2 comprises a coupling site 5 connecting the dressing 1 to the negative pressure source 102.

FIG. 1 furthermore conceptually illustrates a negative wound therapy (NPWT) system 100. The NPWT system 100 comprises a negative pressure device 101 comprising a negative pressure source 102 and a canister 103 for collecting fluids.

The NPWT system 100 furthermore comprises a tubing assembly 104, configured to fluidly connect the NPWT dressing 1 to the NPWT pressure source 102.

The negative pressure source 102 is a negative pressure pump adapted for establishing a negative pressure when the negative pressure pump is in an active state. The negative pressure pump may be any type of pump that is biocompatible and maintains or draws adequate and therapeutic vacuum levels. Preferably, the negative pressure level to be achieved is in a range between about −20 mmHg and about −300 mmHg. In embodiments of the present disclosure, a negative pressure range between about −80 mmHg and about −180, preferably between about −100 and −150 mmHg, more preferably between −110 and −140 mmHg is used. In embodiments, the negative pressure pump is a pump of the diaphragmatic or peristaltic type.

The tubing assembly 104 is connected to a backing layer 2 of the NPWT dressing at a coupling site 5, optionally via a coupling member. The tubing assembly 103 comprises a first tubing 105 configured to be connected to the NPWT dressing 1, wherein a distal end 107 of the first tubing 105 is attached to a connector unit 108. A second tubing 109 is connected to the canister 103 and to the connector unit 108. The system furthermore comprises means to supply air to the NPWT dressing 1.

During use, the NPWT dressing 1 is arranged at a wound site of the user/patient, forming a sealed space. The tubing (105 and 109) is provided to fluidly connect the NPWT dressing 1 to the negative pressure device 101. The NPWT device 101 is then activated, e.g. by the user/patient, by pressing the start/pause button. The negative pressure pump 102 is thereby activated. When activated, the negative pressure pump will start to evacuate air through the canister 103, the tubing (105 and 109) and the sealed space formed by the dressing 1. Accordingly, the negative pressure will be created within the sealed space. In case a liquid has been formed at the wound site, this liquid from the wound site may at least partly be “drawn” from the wound site, through the tubing (105 and 109), and into the canister 103. The amount of liquid; i.e. exudate that is drawn from the wound and collected in the canister 103 will depend on the type of wound that is being treated as well as the type of wound dressing used.

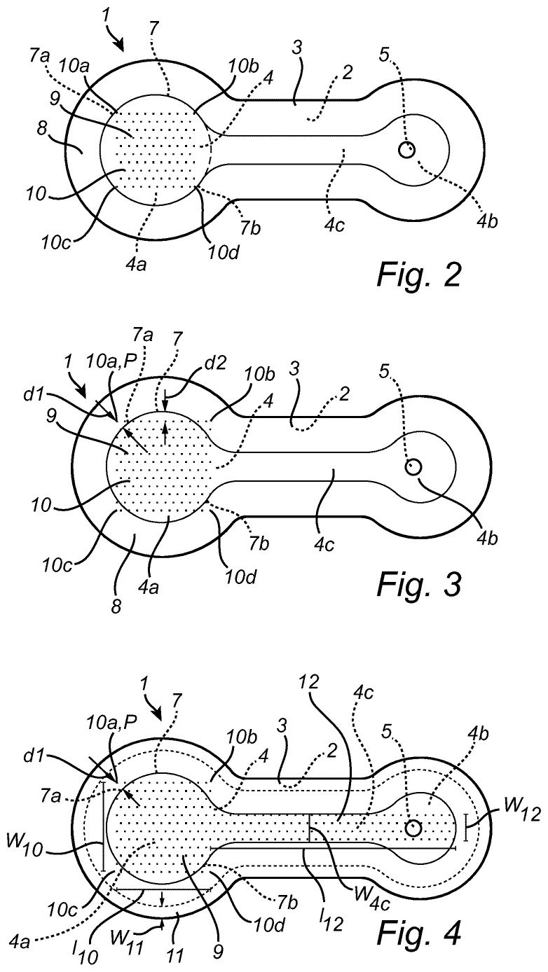

FIG. 2 illustrates a NPWT dressing 1 comprising a backing layer 2, an adhesive skin contacting layer 3 and an absorbent pad 4 arranged between the backing layer 2 and the adhesive skin contacting layer 3. The backing layer 2 comprising a coupling site 5 configured to connect the dressing 1 to a negative pressure source 102, as illustrated in FIG. 1. The absorbent pad 4 has a pad outer edge(s) 7 and the backing layer 2 and the adhesive skin contacting layer 3 extend beyond the pad outer edge(s) 7 forming a border portion 8 around the absorbent pad 4. In this Figure, the backing layer 2 and the adhesive skin contacting layer 3 are coextensive.

The NPWT dressing 1 illustrated in FIG. 2 is suitable to use for a diabetic foot ulcer, as disclosed in FIG. 1 and the absorbent pad 4 has a wound facing portion 4a, a negative pressure source connecting portion 4b and a bridging portion 4c connecting the wound facing portion 4a with the negative pressure source connecting portion 4b. The wound facing portion 4a has a circular shape. Alternatively, the wound facing portion may have a oval shape. When defining the boundary of the wound facing portion 4a towards the bridging portion 4c, the boundary may be seen as a continuation of the circular shape, or alternatively the oval shape, such as forming a complete symmetrical circle, as illustrated by the dotted line.

The backing layer 2 of the dressing 1 may comprise at least one film. For example, it may comprise more than one film. The one or more films may comprise a thermoplastic elastomer. A thermoplastic elastomer may be stretched to moderate elongations, and upon the removal of stress, return to its original shape. Examples of suitable materials comprising thermoplastic elastomer include polyurethane, polyamide and polyethylene. The backing layer may also be a laminate of polyester based nonwoven materials and at least one polyurethane film.

Preferably, the backing layer comprises a polyurethane film.

The thickness of the backing layer may be in the range of from 10 to 40 pm, preferably from 15 to 30 pm.

The adhesive skin contact layer may comprise a silicone-based adhesive, the adhesive skin contact layer may alternatively comprise a polymeric film provided with a silicone based adhesive coating.

The dressing 1 is illustrated with the side facing a patient during use, i.e., the side provided with the adhesive skin contacting layer 3. The adhesive skin contacting layer 3 comprises perforations 9. The perforations 9 form a first perforated area 10 extending over the absorbent pad 4 allowing passage of liquid from a wound into the absorbent pad 4. The absorbent pad 4 has a non-rectangular shape and the first perforated area 10 forms a rectangular shape having a first, a second, a third and a fourth corner portion 10a,10b,10c,10d.

When defining the boundary of the first perforated area 10, an imaginary line is drawn between the outermost perforations, from an outermost point of the respective perforation, thereby defining the first perforated area within the boundary formed.

The outer edge 7 of the absorbent pad 4 has a first outer edge portion 7a and a second outer edge portion 7b. The first and the second outer edge portion 7a,7b are opposing outer edge portions and the first corner portion 10a of the first perforated area 10 is arranged at the first outer edge portion 7a and the fourth corner portion 10d is arranged at the second outer edge portion 7b of the absorbent pad 4, or at least the respective first and fourth corner portions 10 a,10d is arranged at a distance d1 of 10 mm or less from the first and the second outer edge portion 7a,7b respectively.

Preferably, the respective first and the fourth corner portion 10a,10d is arranged at a distance di of 7 mm or less from the first and the second outer edge portion 7 a,7b respectively. In the exemplary embodiment of FIG. 2, the first to fourth corner portions 10 a-10d are arranged with a distance of 2 mm or less from the outer edge 7 of the absorbent pad.

The perforations 9 in the first perforated area 10 are evenly distributed over the entire first perforated area 10 and the perforations 9 are arranged with a distance within the range of from 1 to 4 mm to an adjacent perforation 9.

The first perforated area 10 may cover at least 80% of the wound facing portion 4 a, which ensures a high liquid drainage from the wound site.

FIG. 3 illustrates a NPWT dressing 1 according to the present disclosure which, like the dressing 1 illustrated in FIG. 2, is suitable for use for a diabetic foot ulcer and comprises a wound facing portion 4a, a negative pressure source connecting portion 4b and a bridging portion 4c connecting the wound facing portion 4a with the negative pressure source connecting portion 4b. The wound facing portion 4a has a circular or oval shape.

The NPWT dressing 1 comprises a backing layer 2, an adhesive skin contacting layer 3 and an absorbent pad 4 arranged between the backing layer 2 and the adhesive skin contacting layer 3. The backing layer 2 comprises a coupling site 5 connecting the dressing 1 to a negative pressure source 20, as illustrated in FIG. 1.

The adhesive skin contacting layer 3 comprises perforations 9. The perforations 9 form a first perforated area 10 extending over the absorbent pad 4 allowing passage of liquid from a wound into the absorbent pad 4. The absorbent pad 4 has a non-rectangular shape and the first perforated area 10 forming a rectangular shape having a first, a second, a third and a fourth corner portion 10a,10b,10c,10d.

The perforations 9 in the first perforated area 10 are evenly distributed over the entire first perforated area 10 and the perforations 9 are arranged with a distance within the range of from 1 to 4 mm to an adjacent perforation 9. The first perforated area 10 may cover 85% or more of the wound facing portion 4 a, which ensures a high liquid drainage from the wound site.

In the exemplary embodiment shown in FIG. 3, the first perforated area 10 extends into the border portion 8 around the absorbent pad 4 with a greatest distance di between an outmost point p of the first perforated area 10, corresponding to the first corner portion 10 a, and the pad outer edge(s) 7 being within the range of from 0.5 mm and 20 mm, preferably within the range of from 0.5 mm and 10 mm.

Furthermore, the first corner portion 10a is arranged at the first outer edge portion 7a and the fourth corner portion 10d is arranged at the second outer edge portion 7b and a greatest distance d2, as measured from a point arranged between the first and the second corner portions 10a-10b, from the first perforated area 10 and the pad outer edge(s) 7 is within the range of from 0.5 mm to 15 mm.

A ratio of the distance d1 and the distance d2 is within the range of from 0.2:1 to 1:1. FIG. 4 illustrates the same type of NPWT dressing 1 as illustrated in FIGS. 2 and 3 which is suitable for use for a diabetic foot ulcer. The NPWT dressing 1 comprises a wound facing portion 4a, a negative pressure source connecting portion 4b and a bridging portion 4c connecting the wound facing portion 4a with the negative pressure source connecting portion 4b. The wound facing portion 4a has a circular or oval shape.

The NPWT dressing 1 comprises a backing layer 2, an adhesive skin contacting layer 3 and an absorbent pad 4 arranged between the backing layer 2 and the adhesive skin contacting layer 3. The backing layer 2 comprises a coupling site 5 connecting the dressing 1 to a negative pressure source 20, as illustrated in FIG. 1.

The adhesive skin contacting layer 3 comprises a first perforated area 10 extending over the absorbent pad 4 allowing passage of liquid from a wound into the absorbent pad 4. The first perforated area 10 has a rectangular shape having a first, a second, a third and a fourth corner portion 10a,10b,10c,10d.

The first perforated area 10 extends into the border portion 8 around the absorbent pad 4 with a greatest distance di between an outmost point p of the first perforated area 10 and the pad outer edge(s) 7 being within the range of from 1 mm and 10 mm.

In the exemplary embodiment illustrated in FIG. 4, the border portion 8 comprises a circumferential perforation free area 11 extending along an outer edge of the entire NPWT dressing 1. A width w11 of the circumferential perforation free area 11 is at least 10 mm, preferably at least 15 mm. Since this type of NPWT dressing 1 is intended to be applied to a foot, ankle and lower part of the leg, the dressing will be exerted to torsional forces upon movement of the foot. It is therefore important to have a high adhesion along the entire boarder portion to avoid that air passages are formed such that air enters into the absorbent pad and affecting the pressure and the liquid drainage negatively. The circumferential perforation free area has been found to provide such advantages.

In the NPWT dressing 1 illustrated in FIG. 4, the adhesive skin contacting layer 3 furthermore comprises a second perforated area 12, the second perforated area 12 extending over the bridging portion 4c and over the negative pressure source connecting portion 4b of the absorbent pad 4. This may have the advantage of making the dressing slightly more versatile while not impeding on the manufacturing process.

The first perforated area 10 has a width w10 and a length l10 and the second perforated area 12 has a width w12 and a length l12. The width w10 of the first perforated area 10 is greater than the width w12 of second perforated area 12, optionally at least 2 times greater. Furthermore, the length l12 of the second perforated area 12 is greater than the length l10 of first perforated area 10, optionally at least 2 times greater.

The bridging portion 4c may have a width w4a and a ratio of the width w12 of the second perforated area 12 and the width w4c of the bridging portion 4c may be within the range of from 1:1 to 1:3. Such ratio between the width w4c of the bridging portion portion 4c and the width w₁₂ of the second perforated area 12 has been found advantageous in that the need for liquid drainage is lower in the bridging portion 4c, while still allowing some liquid drainage if a smaller amount of liquid from the wound has leaked into this portion.

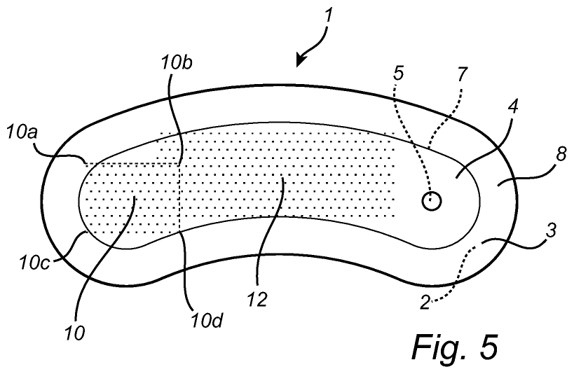

FIG. 5 illustrates a further embodiment of an NPWT dressing 1 comprising a backing layer 2, an adhesive skin contacting layer 3 and an absorbent pad 4 arranged between the backing layer 2 and the adhesive skin contacting layer 3. The backing layer 2 comprises a coupling site 5 configured to connect the dressing 1 to a negative pressure source 102, as illustrated in FIG. 1. The absorbent pad 4 has a pad outer edge(s) 7 and the backing layer 2 and the adhesive skin contacting layer 3 extend beyond the pad outer edge(s) 7 forming a border portion 8 around the absorbent pad 4. The absorbent pad 4 has a bent elongated shape and may be suited for diabetic foot ulcers and general pressure ulcers especially on protruding areas of the body, although applications to other wounds are possible.

The adhesive skin contacting layer 3 comprises perforations 9. The perforations 9 form a first perforated area 10 extending over the absorbent pad 4 allowing passage of liquid from a wound into the absorbent pad 4. The adhesive skin contacting layer 3 furthermore comprises a second perforated area 12 extending over the absorbent pad 4 allowing passage of liquid from a wound 11 into the absorbent pad 4. The first and second perforated areas 10,12 are overlapping and therefore each of the first and the second perforated area 12 has a respective rectangular shape.

In general, the perforated area(s) can also be described as two, or more, rectangular shapes merged together, sharing the overlapping area, or optionally being positioned adjacent each other. It should be noted that although the rectangular shapes have different sizes, the perforations preferably extend seamlessly between the first and the second perforated area. In this respect, the rows, and/or lines, of perforations can extend as uninterrupted rows, and/or lines, of perforations with the same distance between the perforations. The term uninterrupted should hereby be understood to mean that there are no variations of the pattern of perforations in the line or rows.

In the dressing 1 illustrated in FIG. 5, the section of the adhesive skin contacting layer 3 coinciding with the coupling site 5 is non-perforated.

When defining the boundary of the first perforated area 10, an imaginary line is drawn between the outermost perforations, from an outermost point of the respective perforation, thereby defining the first perforated area 10 within the boundary formed. Furthermore, when defining the boundary of the first perforated area 10 and the second perforated area 12, the boundary may be seen as a continuation of the respective rectangular shape such as forming a complete symmetrical rectangle. For illustrative purposes, the boundaries of the first perforated area 10 with a first, a second, a third and a fourth corner portion 10a,10b,10c,10d is shown in FIG. 5.

Even though the present disclosure has been described with reference to specific exemplifying embodiments thereof, many different alterations, modifications and the like will become apparent for those skilled in the art.

Variations to the disclosed embodiments can be understood and effected by the skilled addressee in practicing the present disclosure, from a study of the drawings, the disclosure, and the appended claims. Furthermore, in the claims, the word “comprising” does not exclude other elements or steps, and the indefinite article “a” or “an” does not exclude a plurality.

It is also disclosed herein a wound dressing comprising a wound pad arranged between a skin contacting layer and a backing layer. The wound pad having at least a portion which is non-rectangular, preferably oval or a circular periphery. The skin contacting layer has a perforation area having a rectangular shape as disclosed herein.

1. A negative pressure wound therapy (NPWT) dressing comprising a backing layer an adhesive skin contacting layer and an absorbent pad arranged between the backing layer and the adhesive skin contacting layer, the backing layer comprising a coupling site configured to connect the dressing to a negative pressure source, the absorbent pad having a pad outer edge(s), the backing layer and the adhesive skin contacting layer extending beyond the pad outer edge(s) forming a border portion around the absorbent pad, the adhesive skin contacting layer comprising perforations, the perforations forming a first perforated area extending over the absorbent pad allowing passage of liquid from a wound into the absorbent pad, the absorbent pad having a non-rectangular shape and the first perforated area forming a rectangular shape having a first, a second, a third and a fourth corner portion.

2. The NPWT dressing according to claim 1, wherein the pad outer edge has a first outer edge portion and a second outer edge portion, the first and the second outer edge portion being opposing outer edge portions and wherein the first corner portion of the first perforated area is arranged at the first outer edge portion and the fourth corner portion is arranged at the second outer edge portion of the absorbent pad, or at least the respective first and the fourth corner portion is arranged at a distance of 10 mm or less from the first and the second outer edge portion respectively.

3. The NPWT dressing according to claim 1, wherein the pad outer edge(s) has/have a first outer edge portion and a second outer edge portion, the first and the second outer edge portion being opposing outer edge portions and wherein the first perforated area extends from the first outer edge portion to the second outer edge portion of the absorbent pad and wherein the first perforated area extends into the border portion around the absorbent pad with a greatest distance d1 between an outmost point (p) of the first perforated area and the pad outer edge(s) being within the range of from 0.5 mm and 10 mm.

4. The NPWT dressing according to claim 1, wherein and the first corner portion is arranged at the first outer edge portion and the fourth corner portion is arranged at the second outer edge portion and wherein a greatest distance d2 from the first perforated area and the pad outer edge(s) is within the range of from 0.5 mm to 15 mm, as measured from a point arranged between the first and the second corner portions.

5. The NPWT dressing according to claim 3, wherein a ratio of the distance d1 and the distance d2 is within the range of from 0.2:1 to 1:1.

6. The NPWT dressing according to claim 1, wherein the border portion comprises a circumferential perforation free area extending along an outer edge of the entire NPWT dressing and wherein the circumferential perforation free area has a width (w) of at least 10 mm.

7. The NPWT dressing according to claim 1, wherein the perforations in the first perforated area are arranged with a distance within the range of from 1 to 4 mm to an adjacent perforation.

8. The NPWT dressing according to claim 1, wherein the perforations are evenly distributed over the entire first perforated area.

9. The NPWT dressing according to claim 1, wherein the absorbent pad has a wound facing portion, a negative pressure source connecting portion and a bridging portion connecting the wound facing portion with the negative pressure source connecting portion and wherein the first perforated area is arranged over the first wound facing portion.

10. The NPWT dressing according to claim 9, wherein the wound facing portion has a circular or oval shape.

11. The NPWT dressing according to claim 10, wherein the first perforated area covers at least 80% of the wound facing portion.

12. The NPWT dressing according claim 9, wherein the adhesive skin contacting layer comprises a second perforated area and wherein the second perforated area extend over the bridging portion of the absorbent pad.

13. The NPWT dressing according to claim 12, wherein the first perforated area has a width (w1) and a length (l1) and the second perforated area has a width (w2) and a length (l2) and wherein the width (w1) of the first perforated area is greater than the width (w2) of second perforated area, optionally at least 2 times greater.

14. The NPWT dressing according to claim 1, wherein the absorbent pad has an elongated non-rectangular shape and wherein the adhesive skin contacting layer comprises a second perforated area extending over the absorbent pad allowing passage of liquid from a wound into the absorbent pad, the second perforated area having a rectangular shape.

15. The NPWT dressing according to claim 14, wherein the second perforated area is arranged offset with respect to the first perforated area as seen along any transverse axis extending in a transverse direction (T).

16. The NPWT dressing according to claim 1, wherein said adhesive skin contact layer comprises a silicone-based adhesive.

17. The NPWT dressing according to claim 1, wherein said adhesive skin contact layer comprises a polymeric film provided with a silicone based adhesive coating.
